(19) United States
(12) Patent Application Publication
(10) Pub. No.: US 2005/0080570 A1
(43) Pub. Date: Apr. 14, 2005

(54) Predicting probabilities of achieving a desired minimum trough level for an anti-infective agent

(76) Inventor: Edward P. Acosta, Birmingham, AL (US)

Correspondence Address: NEEDLE & ROSENBERG, P.C., SUITE 1000, 999 PEACHTREE STREET, ATLANTA, GA, 30309-3915, US

(21) Appl. No.: 10/942,495

(22) Filed: Sep. 15, 2004

Related U.S. Application Data
(60) Provisional application No. 60/503,054, filed on Sep. 15, 2003.

(51) Int. Cl. G06F 19/00; G01N 33/48; G01N 33/50

(52) U.S. Cl. 702/19

(57) ABSTRACT

A method and system for providing a drug regimen for a patient that considers both pharmacokinetic and drug resistance testing information to reduce the probability that a drug will become ineffective during treatment due to viral susceptibility to infection. The system uses multiple concentration-time curves to determine trough levels for a drug or drugs, which can be used to determine an appropriate drug regimen for the patient.

### FIELD OF THE INVENTION

The present invention relates to calculating a probability of achieving desired trough concentrations for dosing regimens using anti-infective drugs.

### BACKGROUND OF THE INVENTION

Over the past several years, it has become clear that achieving and maintaining adequate antiretroviral drug concentrations is required to produce a durable virologic response. In fact, many studies are either ongoing or in development that will explore therapeutic drug monitoring (TDM) of antiretrovirals in naive and experienced human immunodeficiency virus (HIV)-infected patients. The classic method of applying TDM uses measured drug concentrations to alter a dosing regimen in order to achieve a targeted level of exposure. However, a primary concern when utilizing this method is choosing the appropriate target concentration.

Some in vivo clinical pharmacodynamic data are available for various antiretrovirals, mostly protease inhibitors (PIs), but efficiently collecting these data is difficult.

Recently, in vitro phenotypic sensitivity testing of individual patients' viral isolates has become available, enabling clinicians to use susceptibility as a surrogate for concentration-response data. Even so, clinical use of TDM in this country has not been widely used. This is likely due to the logistics of sample collection, difficulty in interpretation of results, and lack of large, prospective studies clearly defining the role of TDM.

As a result, alternative methods of incorporating antiretroviral pharmacokinetics into therapeutic decisions are being explored. The method gaining the most attention is the inhibitory quotient (IQ), or the Cₘᵢₙ/IC ratio, where Cₘᵢₙ is the measured trough concentration of a particular drug in a patients' regimen and IC is the in vitro drug inhibitory concentration required to block 50%, 90%, or 95% of viral replication (typically the IC50, IC90, or IC95). Several studies have attempted to define the optimal Cmin/IC50 ratio required to produce long-term viral suppression, and the ratio has been linked to clinical outcome.

Although the IQ concept is appealing, its applicability in the clinic setting remains untested and undefined. There is still a growing clinical need for the development of predictive methods that incorporate pharmacokinetic and viral resistance testing data in order to better manage treatment-experienced patients.

### SUMMARY OF THE INVENTION

The present invention provides a method and system for calculating a probability of achieving desired trough concentrations for a desired dosing regimen, which can be used in managing drug treatments for patients.

The present invention (1) collects pertinent pharmacokinetic parameters for a drug; (2) builds a pharmacokinetic model for each dosing regimen; (3) simulates each dosing regimen model; (4) extracts trough drug levels from each simulation; (5) collects data associated with drug concentrations that reduce viral replication in vitro by a specified percentage; (6) compares the simulated trough levels with a PBIC₅₀ and (7) outputs the probabilities of achieving trough levels at or above the PBIC₅₀.

### BRIEF DESCRIPTION OF THE DRAWINGS

The foregoing and other advantages and features of the invention will become more apparent from the detailed description of exemplary embodiments of the invention given below with reference to the accompanying drawings.

FIG. 1A illustrates a plurality of concentration-time curves for a plurality of dosing regimens;

FIG. 1B illustrates a plurality of concentration-time curves for a plurality of dosing regimens;

FIG. 2 is a spreadsheet illustrating a plurality of dosing regimens and a plurality of simulated trough levels for each drug regimen;

FIG. 3 is a chart illustrating a combination of dosing regimens containing low-dose RTV. APV refers to Amprenavir or fos-Amprenavir; RTV refers to Ritonavir; IDV refers to Indinavir; SQV refers to Saquinavir; LPVr refers to Lopinavir+Ritonavir Combination Product; NFV refers to Nelfinavir; EFV refers to Efavirenz; NVP refers to Nevirapine; ATV refers to Atazanavir; TPV refers to Tipranavir; BID twice daily dosing; QD refers to once daily dosing; and

FIG. 4 is a schematic diagram of a processing system which implements methods in accordance with the present invention.

### DETAILED DESCRIPTION

The present invention integrates expected drug exposure information with in vitro susceptibility testing to calculate the probability of achieving desired trough concentrations, particularly those above a measured protein binding-corrected level, such as IC₉₅ (PBIC₉₅), IC₅₀ (PBIC₅₀), etc. These probabilities can be used to determine which dual PI-based salvage regimen is most likely to be successful in the management of treatment-experienced patients.

Although the present invention can be used to predict the probability of achieving desired trough levels of any suitable anti-infective agent including, for example, anti-bacterial, antibiotic, antifungal and anti-viral, in exemplary embodiments of the invention described below the anti-infective agent is an anti-viral drug (e.g., an anti-retroviral drug) or combination of such drugs. In particular, it is believed that integration of pharmacokinetic and viral drug resistance testing information maximizes the benefit of antiretroviral therapy in the management of highly treatment-experienced HIV-infected subjects.

The method can be practiced using a suitable computer such as a personal computer, hand-held computer or PDA. As described below, portions of the software involved can be obtained from commercial sources or can readily be produced by persons skilled in the art to which the invention relates in view of the teachings in this patent specification. In an exemplary embodiment of the invention, which relates to antiretroviral drugs, the steps of the method are as follows:

In a first step (1) of the method, pertinent pharmacokinetic parameters for the drug or drugs are collected. Such data are available from published literature with which persons skilled in the art are familiar. These data include parameters such as area-under-concentration/time-curve (AUC), oral clearance (CL/F), maximum concentration (Cₘₐₓ), terminal distribution volume (V/F), and others that may be available. These are collected for any regimen where protease inhibitors are used, whether in combination therapy, alone, or with other classes of antiretroviral drugs.

In a second step (2) of the method, a pharmacokinetic model is built for each regimen. The parameters collected from step 1 are used to create a pharmacokinetic model. Either a one-compartment model or two-compartment model is suitable. As known by persons skilled in the art, commercially available software can be used to build the model. For example, the ADAPT II (release 4) software package available from Biomedical Simulations Resource, Los Angeles, Calif. can be used.

One-compartment model parameters from step (1) can include: oral clearance (CL/F), terminal distribution volume (V₂/F), absorption rate constant (kₐ), and absorption lag phase (t_{lag}). Two-compartment parameters can include: total (CL/Fₜₒₜ) and distributional (CL/F_{d}) clearance, central (V_{c}/F) and peripheral (Vₚ/F) distribution volumes, and kₐ and t_{lag}.

To account for intersubject pharmacokinetic variability, all clearance and distribution volumes were assigned a coefficient of variation (CV) of 50%. A CV of 50% was also applied to ka and tlag. Standard deviations can be used if obtained in step (1), but it is suitable to use fixed values that accurately represent the true variability across all regimens studied. These CVs may vary though depending upon the drug(s) being studied. A linear standard deviation variance model can be used to describe additional assay variability.

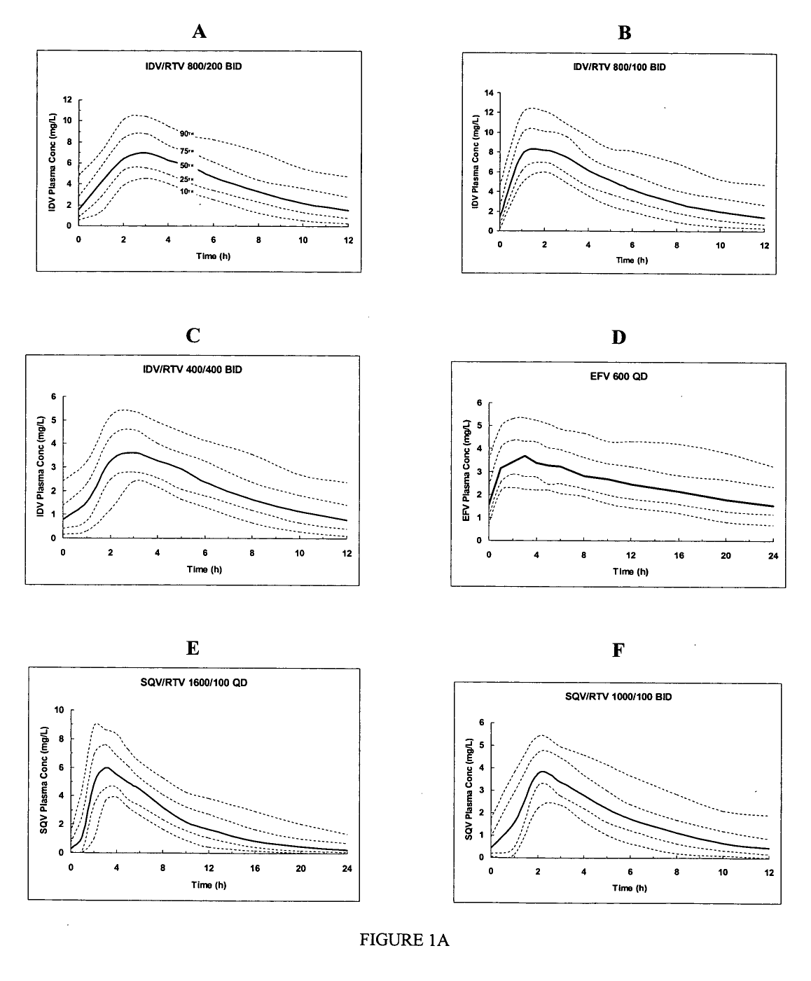

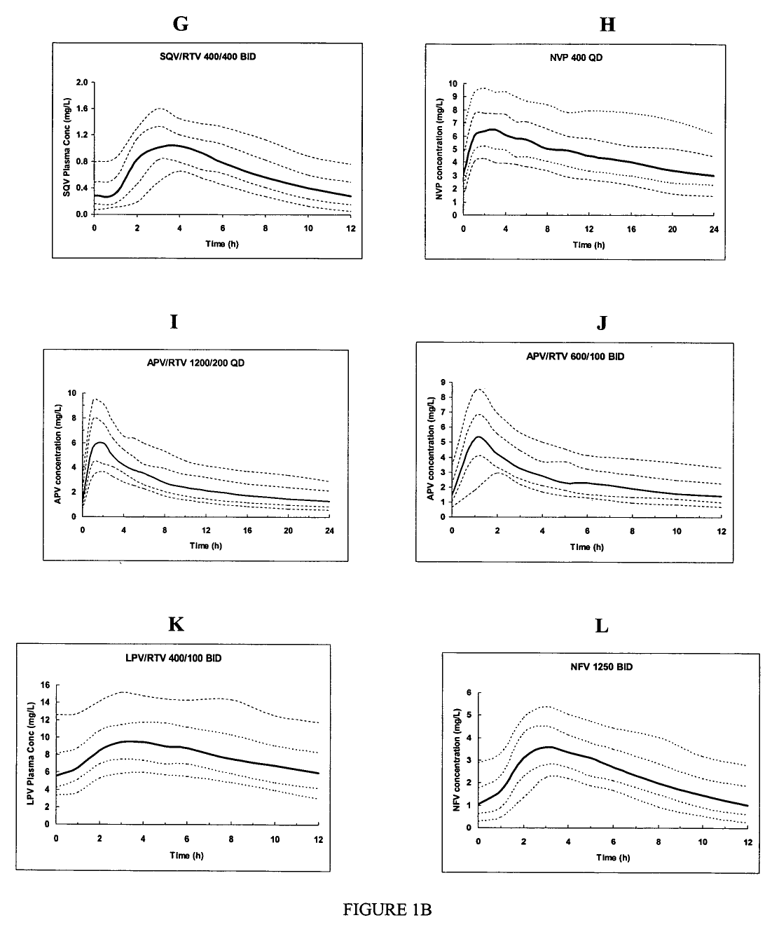

In a third step (3) of the method, a suitable number of population simulations are performed on each regimen model using a suitable simulation method such as Monte Carlo. This technique calculates multiple scenarios of a model by repeatedly sampling pharmacokinetic parameters from the probability distributions and using those values to generate the concentration-time curves. For example, 100 Monte Carlo simulations can be performed on each model, resulting in the generation of 100 corresponding concentration versus time curves. This is analogous to having 100 patients come into a clinic, take a dose or doses of the drugs of interest, and measuring their blood levels over the course of the dosing interval. At least about 100 simulations is believed to be suitable for purposes of this exemplary embodiment of the invention, but a larger number of simulations (e.g., 1000) would not significantly alter the range of resulting pharmacokinetic parameters and concentration-time curves. These concentration-time data can be used to produce percentile curves, such as the 10th, 25th, 50th (median), 75th, and 90^{th}, for each dosing regimen, as illustrated in FIGS. 1A and 1B.

For each regimen, the 10^{th} to 90^{th} percentile concentration-time curves are shown, demonstrating the inherent interpatient variability of the drugs of interest. For example, even Efavirenz (EFV), which has a long half-life, has trough concentrations ranging from approximately 600-700 ng/mL (10^{th} percentile) to upwards of 4000 ng/mL (90^{th} percentile).

These simulated concentration-time curves serve two important purposes. The first purpose is to aid in the interpretation of antiretroviral plasma drug concentrations. Each plot shown above shows the median concentration-time curve and the variability around that curve. Therefore, if a plasma sample was collected from an individual patient for drug concentration determination, these figures will allow the clinician or other health care professional to determine how that patient compares with the average population. A second purpose is to provide an additional tool for optimization of salvage therapy when used in conjunction with viral drug phenotypic data.

In a fourth step (4) of the method, the range of trough drug levels is extracted from the concentration-time curves for each regimen. Since 100 simulations were performed for each regimen, there are 100 trough drug levels available from each regimen. The trough drug level is the lowest amount of drug in the body just before the next dose is taken. So, for a regimen that is administered every 12 hours, the trough level will generally occur 12 hours after the last dose. The trough levels are important because research has shown strong relationships between trough levels (for protease inhibitors and NNRTIs like efavirenz) and clinical outcome, i.e., low trough levels cause viral resistance to develop and ultimately the drug regimen will fail.

In a fifth step (5) of the method, data representing drug concentration that reduces viral replication in vitro by a specified percentage are collected. ViroLogic, Inc. of San Francisco, Calif. is a commercial source of data representing drug concentration that reduces viral replication in vitro by 50% (IC₅₀) (ViroLogic's PHENOSENSE™ report), but the data could be obtained in any other manner or from any other source that may provide it. The IC₅₀ represents how resistant a virus may be; the higher the IC₅₀ the more drug it will take to prevent viral replication.

When using an IC₅₀ to target plasma drug levels, it should be adjusted to account for plasma protein binding. The IC₅₀ data are corrected for protein binding to produce a PBIC₅₀. Any suitable method known in the art can be used to correct IC₅₀s for protein binding. Several methods include dividing the IC₅₀ by the known free-fraction of drug, quantitating the free drug level in plasma, and determining a protein binding correction factor based on in vitro protein binding studies using 50% human serum (HS).

Several well-conducted studies examining this issue have been published, and it is suitable to use the average value across different studies. Using data from some of these studies, and taking IDV as an example, 50% binding would lead to an appropriate correction factor of 2 (i.e. 100 ng/mL divided by 0.5 equals 200 ng/mL). IDV, however, is 60% bound to plasma proteins, so the conversion factor must be slightly higher than 2.0. Results from a 100% HS study state the conversion factor to be 3.5, which is likely an overestimate. The average value across four such studies for IDV is 2.4, which is believed to be a reasonable estimate.

The following table lists as an example the results of four studies involving eight drugs and the average correction factor for each of these drugs:

TABLE 1
PROTEIN-BINDING CORRECTION
FACTORS FOR PIs AND NNRTIs

| Drug | 50% HS | 50% HS | 50% HS | 100% HS | Average |
|---|---|---|---|---|---|
| IDV | 2 | 2 | 2.1 | 3.5 | 2.4 |
| APV | 6 | 7 | 3.8 | 10.5 | 6.8 |
| LPV | 5 | | 3.9 | 14.2 | 7.7 |
| RTV | 20 | 20 | 10.2 | 29.7 | 20.0 |
| SQV | 25 | 26 | 14.7 | 28.0 | 23.4 |
| NFV | 35 | 37 | 21.9 | 90.6 | 46.1 |
| EFV | | | 11.2 | 25.5 | 18.4 |
| NVP | | | 1.3 | 1.2 | 1.3 |

The average value in the last column for a particular drug is multiplied by the IC₅₀ derived from the PHENOSENSE™ report. In this regard, the IC₅₀ becomes a protein-binding corrected IC₅₀ (PBIC₅₀). It is the PBIC₅₀ that is compared with the range of simulated trough drug levels in order to determine the probability of achieving a trough drug levels above the PBIC₅₀ for each regimen examined. These PBIC₅₀'s can then be compared directly to the simulated concentration-time curves in order to estimate the probability that the trough concentration of a particular drug regimen will be at or above the PBIC₅₀. In other words, one can determine the probability of achieving an IQ of 1.0 for a particular drug regimen.

In a sixth step (6) of the method, the PBIC₅₀ is compared with the simulated trough drug levels. In the exemplary embodiment of the invention, the comparison is performed using a spreadsheet program such as MICROSOFT EXCEL, but in other embodiments this step can be integrated with the other steps in a single software program or can be performed by computer in any other suitable manner. The plasma drug trough levels from the simulated concentration-time data are input to the spreadsheet. The IC₅₀s, in μM units, are entered directly from the ViroLogic PHENOSENSE™ report. As described in further detail below, the spreadsheet automatically converts the IC₅₀s to ng/mL (using standard conversion factors), corrects for protein binding based on the average values listed in Table 1 above, and determines the probability of achieving an IQ of 1.0 (i.e., a Cₘᵢₙ/PBIC₅₀ ratio of 1.0), 2.0, or 5.0. Briefly, all of the plasma drug trough values from the pharmacokinetic simulations for each regimen (FIGS. 1A and 1B) are listed in ascending order and assigned a rank. In this case, the total rank is 100, since 100 simulations were conducted. As described in further detail below, a simple logical test is written in the column next to the troughs such that if the trough value in a particular cell is greater than the PBIC₅₀, its assigned rank is converted to a percentage of the total rank. As an example, it is assumed that the range of 100 simulated troughs for a particular PI in a RTV-enhanced regimen is from 300 to 5000 ng/mL. The PBIC₅₀ for this PI is 1700 ng/mL, and the first trough in the list that is at or above 1700 ng/mL is, for example, rank number 60; thus, 100 (total number of trough levels) minus the rank 60 (which equals the first trough at or above 1700 ng/mL) divided by 100 is 40%. Therefore, there is a 40% probability of achieving a trough level at or above the patient's PBIC₅₀ for this particular dual PI regimen. The same methods are applied to 2× the PBIC₅₀ and 5× the PBIC₅₀ to determine IQs of 2.0 and 5.0, respectively.

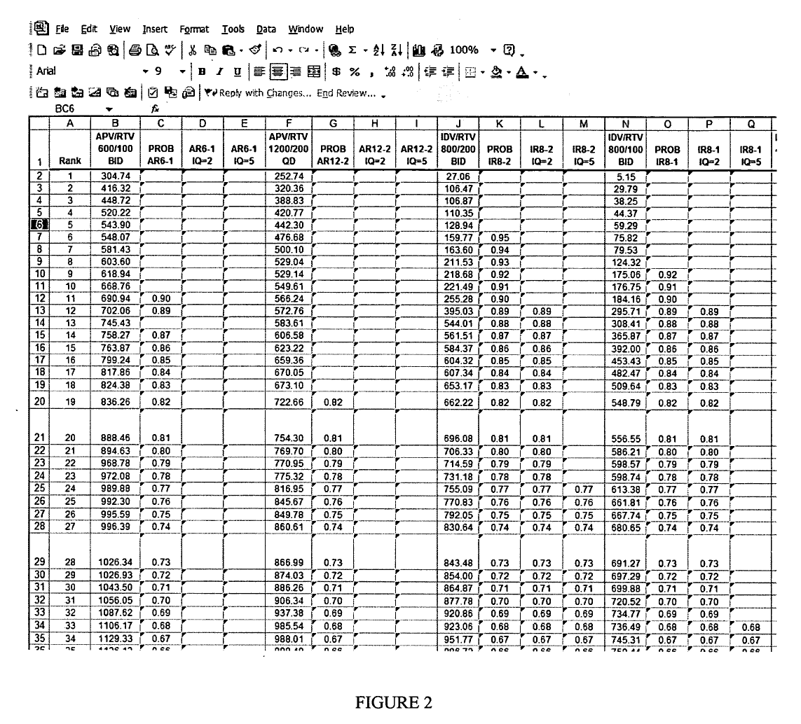

As illustrated in FIG. 2 below, the EXCEL spreadsheet has multiple groups of four columns (columns A-Q being visible in that portion of the spreadsheet screen display shown in FIG. 2). Each group of four columns represents one of the regimens. Row 1 lists the column header labels. Column A is labeled “Rank” and contains a list of numbers from 1 to 100 in ascending order. Therefore, cell A2 (i.e., the cell at column A, row 2) contains the number 1, cell A3 contains the number 2, and so on, with the last cell in column A, cell A101, containing the number 100. The first group of four columns, B-D, relate to a first regimen, the next group of four columns, F-I, relate to a second regimen, and so on.

In the example illustrated in FIG. 2, column B is labeled “APV/RTV 600/100 BID,” and lists the 100 simulated trough levels for this regimen in ascending order. Column C is labeled “PROB AR6-1,” which stands for the probability of the APV/RTV 600/100 BID regimen being greater than the corresponding value in column B. This is the most important column. The formula written in this column is: =IF(B2>$BE$3,(1−(A2−1)/$A$101),“”). Where the cell B2 is the first simulated trough level for this particular regimen, the cell BE3 is where the protein binding corrected IC₅₀ (PBIC₅O) for APV is listed (top section of the output table), the cell A2 is the first rank (i.e. 1) and the cell A101 is the last rank (i.e. 100). This formula then indicates which trough levels listed in column B are above the PBIC50, and lists them in column C. Therefore, the highest value (percentage) in Column C is displayed in the bottom section of the output table under that particular regimen for that particular IQ level. In this example, regimen APV/RTV 600/100 mg BID (IQ=1.0) is displayed in cell AY24. Another formula is written in cell AY24: =MAX(C2:C101), which then calculates the maximum value listed in column C and displays it in cell AY24.

Additional drugs and regimens are contemplated. Examples of various regimens used in the invention include, but are not limited to:
APV/RTV 600/100 BID
APV/RTV 1200/200 QD
IDV/RTV 800/200 BID
IDV/RTV 800/100 BID
IDV/RTV 400/400 BID
SQV/RTV 1600/200 QD
SQV/RTV 1000/100 BID
SQV/RTV 400/400 BID
LPV/RTV 400/100 BID
NFV 1250 BID
EFV 600 QD
NVP 400 QD
ATV-RTV 300/100 QD
LPVr-APV 400/100/600 BID
APV-LPVr 600/400/100BID
ATV/RTV 300/100 mg QD
LPV/RTV/SQV 400/100/1000 mg BID
APV/RTV/IDV 700/100/600 mg BID
SQV/RTV/ATV (multiple doses)
LPV/RTV/ATV (multiple dose)
LPV/RTV/IDV (400/100/600 mg BID
TPV/RTV 500/200 mg BID

To produce an IQ of 2.0 and 5.0, the PBIC₅₀ is multiplied by 2 or 5, respectively. These are displayed in cells BG3 and BG5, respectively. Column D is labeled “AR6-1 IQ=2” and column E is labeled “AR6-1 IQ=5”. The exact same formula is written in these 2 columns as in column C, except the PBIC₅₀ is now from cells BF3 (2×PBIC50) and BG3 (5×PBIC50). Again, the highest percentage listed under columns D and E are displayed in the bottom sections of the output table. For APV/RTV 600/100 mg BID, these correspond to cells AY32 (IQ=2.0) and AY40 (IQ=5.0). The equation: =MAX(D2:D101) is written in cell AY32 and the equation: =MAX(E2:E101) is written in cell AY40, which correspond to the maximum percentages in columns D and E, respectively.

In a seventh step (7) of the method, the probabilities of achieving trough levels at or above the PBIC₅₀ are output to the computer display, as illustrated below with regard to an example in which there are 12 different regimens.

The top section of this exemplary output display lists the drug names (col. 1) and the IC₅₀'s obtained from ViroLogic or other source (col. 2), as a person would enter them into the spreadsheet or other software program at step (5) of the method, described above. In the third column, the spreadsheet or program has converted the IC₅₀ from μM to ng/mL, and in the last column has corrected the IC₅₀ for protein binding (PBIC₅₀).

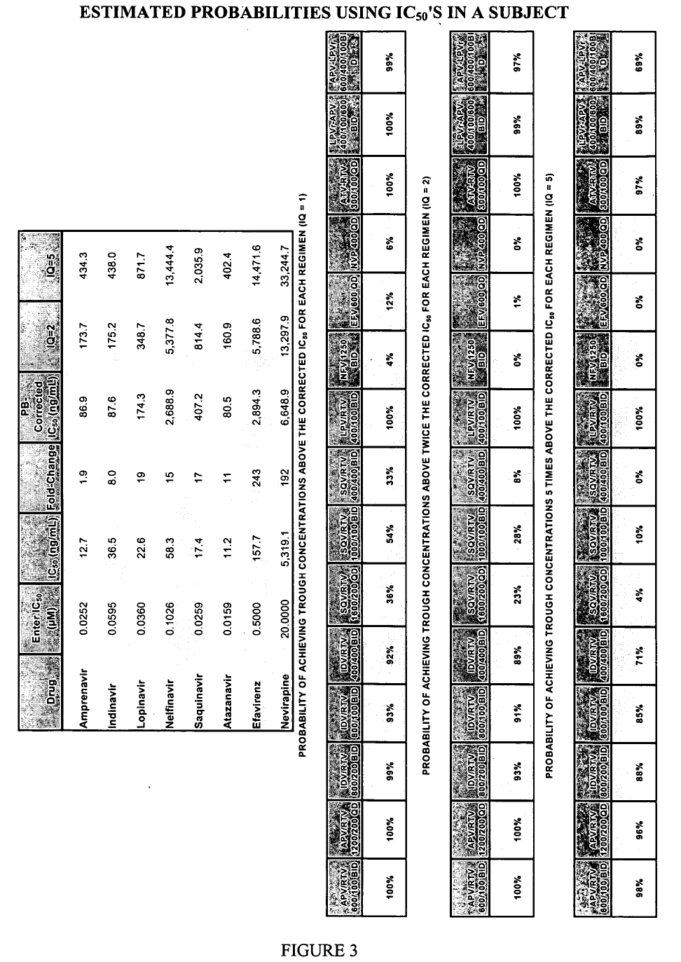

Note that combination regimens containing low-dose RTV are displayed in the output shown in FIG. 3 using only the active protease inhibitor (e.g., there are 3 different dosing regimens for SQV/RTV, but the probability value in the output refers to only SQV) because the combination regimens listed in FIG. 3 all contain ritonavir (RTV) at various dosages, and RTV is not considered an active component of the drug regimen because it is commonly used clinically to enhance or “boost” the plasma drug levels of other drugs, such as other protease inhibitors, in order to achieve significantly higher drug levels relative to a single, unboosted protease inhibitor. Additional dual protease inhibitor plus NNRTI regimens, such as LPV/RTV plus EFV and APV/RTV plus EFV, can be added to the output. For these combination regimens, the probabilities are listed for both active components (LPV and EFV) and displayed as such in the output table.

It is the PBIC₅₀ for each drug that the spreadsheet or other program then compares with the simulated trough levels for each regimen to determine the probability, or likelihood, of achieving an IQ of 1.0. The bottom section of FIG. 3 in this exemplary output display shows the computed percentages. This is the information that a clinician would use to help guide the decision-making process when choosing the next salvage therapy regimen for their patient.

In the illustrated example, the bottom section of FIG. 3 can be interpreted as follows. Both the EFV and NVP regimens would be expected to achieve trough levels above the PBIC₅₀ essentially 100% of the time, even though some viral drug resistance is present. The dual protease inhibitor-containing (PI-containing) regimens of APV/RTV (87 to 93%), IDV/RTV (88-92%), and LPV/RTV (100%) all have high probabilities of achieving trough levels at or above the _{PB}IC₅₀ for each respective enhanced PI regimen at various doses. Others PI regimens, such as SQV/RTV (0-2%) and NFV (1%) have very little chance of achieving the desired trough levels due to the high degree of viral drug resistance present in this subjects isolate. This procedure, which incorporates known pharmacokinetic data and viral drug susceptibility, allows the clinician and patient more choices than the phenotyping alone would provide when determining treatment options for PI and NNRTI-resistant virus.

A clinician can base a decision of the subsequent treatment regimen upon the results computed in accordance with the present invention. For example, a clinician can choose to treat a patient with the regimen having the highest probability. However, as persons skilled in the art to which the invention relates will recognize, other factors may influence the regimen choice, such as past history of the patient (e.g., intolerability to particular drugs). Consequently, a clinician may choose a regimen not having the highest probability. The invention provides the clinician the full range of treatment options available to make an intelligent choice once the clinician has considered both the pharmacokinetics of the drugs and viral susceptibility inherent within this process.

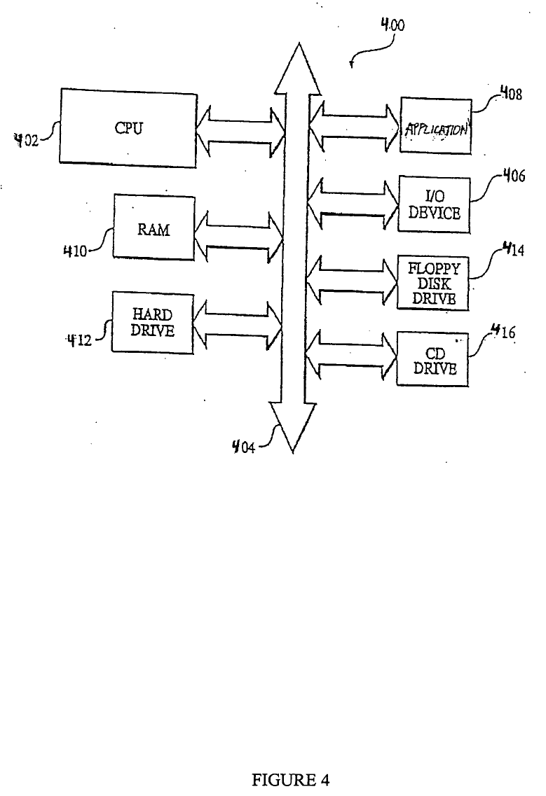

FIG. 4 shows a processor system 400, which includes a computer application 408 that implements methods in accordance with the present invention. Application 408 is used to generate the various concentrations versus time curves and dosing regimens of FIGS. 1-3 for use by a clinician in creating a treatment regimen for a patient. System 400 includes a processor 402 having a central processing unit (CPU) that communicates with various devices over a bus 404. Some of the devices connected to the bus 404 provide communication into and out of the system 400, for example, an input/output (I/O) device 406 and an imager device 408. Other devices connected to the bus 404 provide memory, illustratively including a random access memory (RAM) 410, hard drive 412, and one or more peripheral memory devices such as a floppy disk drive 414 and compact disk (CD) drive 416.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for determining a probability of achieving a desired trough concentration for an anti-infective agent, comprising the steps of:
collecting pharmacokinetic parameters for each of a plurality of drugs;
providing a pharmacokinetic model for each of a plurality of drug regimens for the drugs;
generating a plurality of concentration versus time curves, each corresponding to one of the regimens, using a population simulation in response to the pharmacokinetic models;
identifying a range of trough drug levels from each concentration-time curve;
ranking the identified ranges of trough drug levels;
providing (ICₓₓ) data representing drug concentration reducing pathogen replication by a predetermined percentage (xx) for each drug;
correcting the ICₓₓ for each drug by a correction factor to produce protein binding-corrected (PBICₓₓ) data;
comparing the ranked ranges of trough drug levels to the PBICₓₓ for the drug; and
calculating a probability for the trough drug levels greater than or equal to the PBICₓₓ for the drug.

2. The method of claim 1, wherein the drugs are anti-viral drugs.

3. The method of claim 1, wherein the population simulation is Monte Carlo.

4. The method of claim 1, wherein ICₓₓ is IC₅₀ and PBICₓₓ is PBIC₅₀.

5. The method of claim 1, wherein the pharmacokinetic model is a one-compartment model.

6. The method of claim 1, wherein the pharmacokinetic model is a two-compartment model.

7. The method of claim 1, wherein the plurality of concentration versus time curves is used to create a salvage therapy for a patient.

8. The method of claim 1, wherein the calculated probability of trough levels are used to create a drug regimen for a patient.

9. The method of claim 8, wherein the drug regimen has an inhibitory quotient equal to 1.

10. The method of claim 8, wherein the drug regimen has an inhibitory quotient equal to 2.

11. The method of claim 8, wherein the drug regimen has an inhibitory quotient equal to 5.

12. The method of claim 1 further comprising outputting the calculated probability of trough levels to an end user.

13. The method of claim 1, wherein the comparison step is performed using a spreadsheet program.

14. A method for choosing an anti-viral drug regimen, comprising the steps of:
collecting pharmacokinetic parameters for each of a plurality of anti-viral drug regimens;
providing a pharmacokinetic model for each of the plurality of anti-viral drug regimens;
generating a plurality of concentration versus time curves, each corresponding to one of the regimens, using a population simulation in response to the pharmacokinetic models;
identifying a range of trough drug levels from each concentration-time curve;
ranking the identified ranges of trough drug levels;
providing (ICₓₓ) data representing drug concentration reducing pathogen replication by a predetermined percentage (xx) for each anti-viral drug;
correcting the ICₓₓ for each drug by a correction factor to produce protein binding-corrected (PBICₓₓ) data;
comparing the ranked ranges of trough drug levels to the PBICₓₓ for the drug;
calculating a probability for the trough drug levels greater than or equal to the PBICₓₓ for the drug; and
choosing a drug regimen based at least in part upon a regimen corresponding to a highest calculated probability.

15. The method of claim 14, wherein the drugs are anti-viral drugs.

16. The method of claim 14, wherein the population simulation is Monte Carlo.

17. The method of claim 14, wherein ICₓₓ is IC₅₀ and PBICₓₓ is PBIC₅₀.

18. The method of claim 14, wherein the pharmacokinetic model is a one-compartment model.

19. The method of claim 14, wherein the pharmacokinetic model is a two-compartment model.

20. The method of claim 14, wherein the plurality of concentration versus time curves is used to create a salvage therapy for a patient.

21. The method of claim 14, wherein the drug regimen has an inhibitory quotient equal to 1.

22. The method of claim 14, wherein the drug regimen has an inhibitory quotient equal to 2.

23. The method of claim 14, wherein the drug regimen has an inhibitory quotient equal to 5.

24. The method of claim 14 further comprising outputting the drug regimen to an end user.

25. A method for treating HIV-infected treatment-experienced patients, comprising the steps of:
collecting pharmacokinetic parameters for each of a plurality of anti-viral drug regimens;
providing a pharmacokinetic model for each of the plurality of anti-viral drug regimens;
generating a plurality of concentration versus time curves, each corresponding to one of the regimens, using a population simulation in response to the pharmacokinetic models;
identifying a range of trough drug levels from each concentration-time curve;
ranking the identified ranges of trough drug levels;
providing (ICₓₓ) data representing drug concentration reducing pathogen replication by a predetermined percentage (xx) for each anti-viral drug;
correcting the ICₓₓ for each drug by a correction factor to produce protein binding-corrected (PBICₓₓ) data;
comparing the ranked ranges of trough drug levels to the PBICₓₓ for the drug;
calculating a probability for the trough drug levels greater than or equal to the PBICₓₓ for the drug;
comparing viral resistance data with the calculated probabilities;
choosing a drug regimen to be used in the patient based on comparison of differential probabilities across the comparative regimen; and
administering a chosen drug regimen to a patient.

26. The method of claim 25, wherein the drugs are anti-viral drugs.

27. The method of claim 25, wherein ICₓₓ is IC₅₀ and PBICₓₓ is PBIC₅₀.

28. The method of claim 25, wherein the plurality of concentration versus time curves is used to create a salvage therapy for a patient.

29. The method of claim 25, wherein the drug regimen has an inhibitory quotient equal to 1.

30. The method of claim 25, wherein the drug regimen has an inhibitory quotient equal to 2.

31. The method of claim 25, wherein the drug regimen has an inhibitory quotient equal to 5.

32. The method of claim 25 further comprising outputting the drug regimen to an end user.

33. A computer based medium, comprising an application being executable by a computer, wherein the computer executes the steps of:
collecting pharmacokinetic parameters for each of a plurality of drugs;
providing a pharmacokinetic model for each of a plurality of drug regimens for the drugs;
generating a plurality of concentration versus time curves, each corresponding to one of the regimens, using a population simulation in response to the pharmacokinetic models;
identifying a range of trough drug levels from each concentration-time curve;
ranking the identified ranges of trough drug levels;
providing (ICₓₓ) data representing drug concentration reducing pathogen replication by a predetermined percentage (xx) for each drug;
correcting the ICₓₓ for each drug by a correction factor to produce protein binding-corrected (PBICₓₓ) data;
comparing the ranked ranges of trough drug levels to the PBICₓₓ for the drug; and
calculating a probability for the trough drug levels greater than or equal to the PBICₓₓ for the drug.

34. The computer based medium of claim 33, wherein the drugs are anti-viral drugs.

35. The computer based medium of claim 33, wherein the population simulation is Monte Carlo.

36. The computer based medium of claim 33, wherein ICₓₓ is IC₅₀ and PBICₓₓ is PBIC₅₀.

37. The computer based medium of claim 33, wherein the plurality of concentration versus time curves are used to create a salvage therapy for a patient.

38. The computer based medium of claim 33, wherein the calculated probability of trough levels are used to create a drug regimen for a patient.

39. The computer based medium of claim 38, wherein the drug regimen has an inhibitory quotient equal to 1.

40. The computer based medium of claim 38, wherein the drug regimen has an inhibitory quotient equal to 2.

41. The computer based medium of claim 38, wherein the drug regimen has an inhibitory quotient equal to 5.

42. The computer based medium of claim 33 further comprising outputting the calculated probability of trough levels to an end user.

43. A computer based medium, comprising an application being executable by a computer, wherein the computer executes the steps of:
collecting pharmacokinetic parameters for each of a plurality of anti-viral drug regimens;
providing a pharmacokinetic model for each of the plurality of anti-viral drug regimens;
generating a plurality of concentration versus time curves, each corresponding to one of the regimens, using a population simulation in response to the pharmacokinetic models;
identifying a range of trough drug levels from each concentration-time curve;
ranking the identified ranges of trough drug levels;
providing (ICₓₓ) data representing drug concentration reducing pathogen replication by a predetermined percentage (xx) for each anti-viral drug;
correcting the ICₓₓ for each drug by a correction factor to produce protein binding-corrected (PBICₓₓ) data;
comparing the ranked ranges of trough drug levels to the PBICₓₓ for the drug;
calculating a probability for the trough drug levels greater than or equal to the PBICₓₓ for the drug;
choosing a drug regimen based at least in part upon a regimen corresponding to a highest calculated probability.

44. The computer based medium of claim 43, wherein the drugs are anti-viral drugs.

45. The computer based medium of claim 43, wherein the population simulation is Monte Carlo.

46. The computer based medium of claim 43, wherein ICₓₓ is IC₅₀ and PBICₓₓ is PBIC₅₀.

47. The computer based medium of claim 43, wherein the plurality of concentration versus time curves are used to create a salvage therapy for a patient.

48. The computer based medium of claim 43, wherein the drug regimen has an inhibitory quotient equal to 1.

49. The computer based medium of claim 43, wherein the drug regimen has an inhibitory quotient equal to 2.

50. The computer based medium of claim 43, wherein the drug regimen has an inhibitory quotient equal to 5.

51. The computer based medium of claim 43 further comprising outputting the drug regimen to an end user.

52. A computer based medium, comprising an application being executable by a computer, wherein the computer executes the steps of:
collecting pharmacokinetic parameters for each of a plurality of anti-viral drug regimens;
providing a pharmacokinetic model for each of the plurality of anti-viral drug regimens;
generating a plurality of concentration versus time curves, each corresponding to one of the regimens, using a population simulation in response to the pharmacokinetic models;
identifying a range of trough drug levels from each concentration-time curve;
ranking the identified ranges of trough drug levels;
providing (ICₓₓ) data representing drug concentration reducing pathogen replication by a predetermined percentage (xx) for each anti-viral drug;
correcting the ICₓₓ for each drug by a correction factor to produce protein binding-corrected (PBICₓₓ) data;
comparing the ranked ranges of trough drug levels to the PBICₓₓ for the drug;
calculating a probability for the trough drug levels greater than or equal to the PBICₓₓ for the drug;
comparing viral resistance data with the calculated probabilities;
choosing a drug regimen to be used in the patient based on comparison of differential probabilities across the comparative regimen; and
administering a chosen drug regimen to a patient.

53. The computer based medium of claim 52, wherein the drugs are anti-viral drugs.

54. The computer based medium of claim 52, wherein ICₓₓ is IC₅₀ and PBICₓₓ is PBIC₅₀.

55. The computer based medium of claim 52, wherein the plurality of concentration versus time curves are used to create a salvage therapy for a patient.

56. The computer based medium of claim 52, wherein the drug regimen has an inhibitory quotient equal to 1.

57. The computer based medium of claim 52, wherein the drug regimen has an inhibitory quotient equal to 2.

58. The computer based medium of claim 52, wherein the drug regimen has an inhibitory quotient equal to 5.

59. The computer based medium of claim 52 further comprising outputting the drug regimen to an end user.

60. A system for determining a probability of achieving a desired trough concentration for an anti-infective agent comprising:
a computer system including a processor for executing computer code;
a mass storage device for data storage; and
an application being executable by a computer, wherein the computer executes the steps of:
collecting pharmacokinetic parameters for each of a plurality of drugs;
providing a pharmacokinetic model for each of a plurality of drug regimens for the drugs;
generating a plurality of concentration versus time curves, each corresponding to one of the regimens, using a population simulation in response to the pharmacokinetic models;
identifying a range of trough drug levels from each concentration-time curve;
ranking the identified ranges of trough drug levels;
providing (ICₓₓ) data representing drug concentration reducing pathogen replication by a predetermined percentage (xx) for each drug;
correcting the ICₓₓ for each drug by a correction factor to produce protein binding-corrected (PBICₓₓ) data;
comparing the ranked ranges of trough drug levels to the PBICₓₓ for the drug; and
calculating a probability for the trough drug levels greater than or equal to the PBICₓₓ for the drug.

61. The system of claim 60 further comprising outputting the drug regimen to an end user.

62. A system for choosing an anti-viral drug regimen comprising:
a computer system including a processor for executing computer code;
a mass storage device for data storage; and
an application being executable by a computer, wherein the computer executes the steps of:
collecting pharmacokinetic parameters for each of a plurality of anti-viral drug regimens;
providing a pharmacokinetic model for each of the plurality of anti-viral drug regimens;
generating a plurality of concentration versus time curves, each corresponding to one of the regimens, using a population simulation in response to the pharmacokinetic models;
identifying a range of trough drug levels from each concentration-time curve;
ranking the identified ranges of trough drug levels;
providing (ICₓₓ) data representing drug concentration reducing pathogen replication by a predetermined percentage (xx) for each antiviral drug;
correcting the ICₓₓ for each drug by a correction factor to produce protein binding-corrected (PBICₓₓ) data;
comparing the ranked ranges of trough drug levels to the PBICₓₓ for the drug;
calculating a probability for the trough drug levels greater than or equal to the PBICₓₓ for the drug; and
choosing a drug regimen based at least in part upon a regimen corresponding to a highest calculated probability.

63. The system of claim 62, wherein the plurality of concentration versus time curves is used to create a salvage therapy for a patient.

64. The system of claim 62, wherein the drug regimen has an inhibitory quotient equal to 1.

65. The system of claim 62, wherein the drug regimen has an inhibitory quotient equal to 2.

66. The system of claim 62, wherein the drug regimen has an inhibitory quotient equal to 5.

67. The system of claim 62 further comprising outputting the drug regimen to an end user.

68. A system for treating HIV-infected treatment-experienced patients comprising:
a computer system including a processor for executing computer code;
a mass storage device for data storage; and
an application being executable by a computer, wherein the computer executes the steps of:
collecting pharmacokinetic parameters for each of a plurality of anti-viral drug regimens;
providing a pharmacokinetic model for each of the plurality of anti-viral drug regimens;
generating a plurality of concentration versus time curves, each corresponding to one of the regimens, using a population simulation in response to the pharmacokinetic models;
identifying a range of trough drug levels from each concentration-time curve;
ranking the identified ranges of trough drug levels;
providing (ICₓₓ) data representing drug concentration reducing pathogen replication by a predetermined percentage (xx) for each antiviral drug;
correcting the ICₓₓ for each drug by a correction factor to produce protein binding-corrected (PBICₓₓ) data;
comparing the ranked ranges of trough drug levels to the PBICₓₓ for the drug;
calculating a probability for the trough drug levels greater than or equal to the PBICₓₓ for the drug;
comparing viral resistance data with the calculated probabilities;
choosing a drug regimen to be used in the patient based on comparison of differential probabilities across the comparative regimen; and
administering a chosen drug regimen to a patient.

69. The system of claim 62, wherein the plurality of concentration versus time curves is used to create a salvage therapy for a patient.

70. The system of claim 62, wherein the drug regimen has an inhibitory quotient equal to 1.

71. The system of claim 62, wherein the drug regimen has an inhibitory quotient equal to 2.

72. The system of claim 62, wherein the drug regimen has an inhibitory quotient equal to 5.

73. The system of claim 62 further comprising outputting the drug regimen to an end user.
